(19) United States
(12) Patent Application Publication
(10) Pub. No.: US 2012/0014979 A1
(43) Pub. Date: Jan. 19, 2012

(54) USE OF BCL6 INHIBITORS FOR TREATING AUTOIMMUNE DISEASES

(76) Inventor: Alexander Dent, Avon, IN (US)

(21) Appl. No.: 13/182,163

(22) Filed: Jul. 13, 2011

Related U.S. Application Data
(60) Provisional application No. 61/365,106, filed on Jul. 16, 2010.

(51) Int. Cl. A61K 38/17; A61P 19/04; A61P 19/02; A61P 33/00; A61P 11/00; A61P 1/00; A61P 17/00; A61P 3/10; A61P 15/00; A61P 37/00; A61P 5/16; A61P 25/00; A61P 13/12; A61P 13/10; A61P 7/06; A61P 17/06; A61P 1/16; A61P 25/28; A61P 9/00; A61P 1/04; A61K 39/00

(52) U.S. Cl. 424/185.1; 424/184.1

(57) ABSTRACT

The present invention provides a method of treatment of an autoimmune disease, comprising administering an effective amount of a BCL6 inhibitor to an individual in need thereof.

### CROSS REFERENCES TO RELATED APPLICATIONS

The Present Application claims the benefit of U.S. Provisional Patent Application No. 61/365,106, filed Jul. 16, 2010. The content of this U.S. Provisional Patent Application is hereby incorporated herein in its entirety.

### BACKGROUND

CD4 T helper cells are critical for the proper orchestration of the immune response and are essential for helping B cells make high affinity antigen-specific antibody. Follicular helper T (Tfh) cells are a recently characterized subset of CD4 T cells whose role is specifically to help B cells produce antibody, in part by promoting the germinal center reaction. However, deregulated development of Tfh cells can lead to autoimmune disease. Tfh cells are localized to B cell follicles and thus express the chemokine receptor CXCR5. Tfh cells are also characterized by high expression of the transcription repressor BCL6 and secretion of the B cell stimulatory cytokine interleukin (“IL”)-21. Recent data indicates that BCL6 is the master transcriptional regulator for Tfh cells—forced BCL6 expression can induce the Tfh phenotype in T cells, and Tfh cells cannot develop in the absence of BCL6.

CD4 T helper cells can differentiate into several different types of unique effector lineages (Th1, Th2, Th17, Treg, and Tfh; see FIG. 1). Each subset of cells mediates very different types of immune responses in part via the expression of different “signature” cytokines. Thus, Th1 cells produce Interferon-gamma and help to activate macrophages, Th2 cells produce IL-4 and IL-5 and promote allergic and anti-worm responses, Th17 cells produce IL-17 and IL-22 and promote inflammation and anti-bacterial immunity, Treg cells produce IL-10 and transforming growth factor (“TGF”)-beta and down-modulate the immune response, and Tfh cells produce IL-21 and help B cells form germinal centers and produce antibody (1). Development of the different subsets depends upon the cytokines the cells are exposed to during initial activation through the T cell receptor (“TCR”).

Further, the development of each specific CD4 subset requires a lineage-directing master transcription factor. Thus, Th1 cells require Tbet, Th2 cells require GATA3, Th17 cells require RORgammaT and Treg cells require FoxP3 (1). The transcriptional repressor and B cell oncogene BCL6 is expressed at high levels in Tfh (2, 3). Data from a recent study indicates that BCL6 is a master lineage-directing factor for Tfh cells (4). In this recent study, it was shown that forced BCL6 expression in CD4 T cells can strongly induce Tfh function in vivo, and further that BCL6 function is strictly required for the development of Tfh cells in vivo (4). Two other studies have also recently reported that BCL6 is a master regulator of the Tfh lineage (5, 6).

Tfh cells are characterized by high level of expression of the chemokine receptor CXCR5, which binds the chemokine CXCL13 that is expressed in B cell follicles. Thus, CXCL13 acting on CXCR5 promotes migration of Tfh cells to the B cell follicle (1). Tfh cells have an activated effector T cell phenotype and express ICOS, OX40, CD4OL, CD44 and BTLA, and are negative for CCR7 and CD62 (1, 7). Tfh cells have recently been characterized as expressing PD1 and CD200; however, high levels of CXCR5 and decreased levels of SLAM may be the most specific set of markers for Tfh cells (4). In addition, Tfh cells have been found to be characterized by a BCL6-dependent downregulation of P-selectin glycoprotein ligand 1 (PSGL1, a CCL19- and CCL21-binding protein), indicating that, like CXCR5 and PD1 upregulation, modulation of PSGL1 expression is part of the Tfh phenotype (69). Tfh cells have been described for both mouse and human (8-10).

There are two major stages of Tfh function (11). First, “pregerminal center” Tfh cells interact with antigen-activated B cells and promote the major phases of the initial B cell response: B cell clonal expansion, antibody isotype switch, plasma cell differentiation, and the induction of germinal centers. Second, Tfh cells regulate the fate of B cells and the antibody response by interacting with B cells in the germinal center. Thus, Tfh cells are critical for memory B cell and plasma cell development. The key cytokine produced by Tfh cells is IL-21, which is a factor that potently promotes B cell activation and antibody secretion (12-15).

Tfh cells have been shown to be critical for the proper production of antibody, which is a central and vital component of adaptive immunity. At the same time, the over-production of Tfh cells can lead to autoimmunity as Tfh cells help B cells to produce self-reactive antibodies. This has been most clearly observed with sanroque mice, where a mutation in the roquin gene leads to increased ICOS expression, uncontrolled Tfh development and lupus-like autoimmune disease (10, 17).

High-level expression of BCL6 in germinal center T cells had been reported in 1995 (29, 30), but germinal center T cells were poorly understood until the Tfh subset became characterized in the past few years. Three research groups have published papers showing that BCL6 is required for Tfh cell function (4, 5, 6). In particular, it was shown that infecting wild-type CD4 T cells with BCL6 retrovirus (RV) greatly augmented the ability of CD4 T cells to become Tfh cells in vivo (4). This suggests that BCL6 can actively promote development of the Tfh phenotype.

### SUMMARY OF THE INVENTION

In one aspect, the present invention provides a method for inhibiting or stopping abnormal Tfh activity in individuals suffering from an autoimmune disease, comprising administering to an individual in need of such treatment an effective amount of a BCL6 inhibitor.

In another aspect, the present invention provides a method of treatment of an autoimmune disease in an individual in need thereof, comprising administering an effective amount of a BCL6 inhibitor to inhibit non-specific stimulation of B cells thereby ameliorating autoimmune disease symptoms in the individual.

### BRIEF DESCRIPTION OF THE FIGURES

FIG. 1 illustrates differentiation pathways of CD4 T helper cells into different types of unique effector lineages (Th1, Th2, Th17, T-reg, and Tfh), and master transcription factors controlling such pathways.

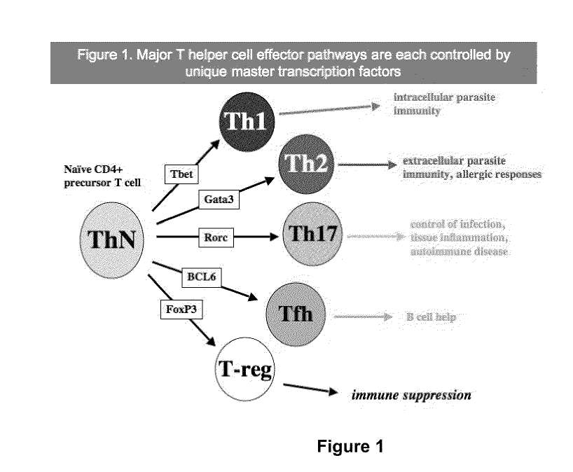

FIG. 2 illustrates a procedure for expressing BCL6 in naïve CD4 T cells and analyzing Tfh function.

FIG. 3 illustrates the results of an experiment studying Tfh gene expression in CD4 T helper cells expressing BCL6.

### DEFINITIONS

As intended herein, the term “peptide” is used in the normal sense to mean a series of residues, typically L-amino acids, connected one to the other typically by peptide bonds between the α-amino and carboxyl groups of adjacent amino acids. Peptides can be synthesized, for example, by solid phase techniques, by recombinant means, or by cleavage from a longer polypeptide. The term includes modified peptides and synthetic peptide analogues, wherein a modification may be made with or without changing the amino acid sequence of the peptide. For example, D-amino acids or other unnatural amino acids can be included, the normal amide bond can be replaced by ester or alkyl backbone bonds, N- or C-alkyl substituents. Glycosylation, lipidation, acetylation, phosphorylation, side chain modifications, constraints such as disulphide bridges and side chain amide or ester linkages can also be included, or any other manipulation or modification, such as conjugation with a labeling component.

As intended herein, “treatment” is an approach for obtaining beneficial or desired clinical results. For purposes of this invention, beneficial or desired clinical results include, but are not limited to, prevention, improvement, lessening severity, reduction, delaying, or ameliorating of any aspect of autoimmune disease, such as inflammation, chronic fever, malaise, joint pains, myalgias, and fatigue.

As intended herein, an “effective amount” of drug, compound, or pharmaceutical composition is an amount sufficient to effect beneficial or desired results including clinical results such as amelioration or reduction in an autoimmune disease. An effective amount can be administered in one or more administrations. For purposes of this invention, an effective amount of drug, compound, or pharmaceutical composition is an amount sufficient to treat, ameliorate, reduce the intensity of and prevent an autoimmune disease. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As intended herein, “ameliorating” an autoimmune disease or one or more symptoms of an autoimmune disease (such as such as inflammation, chronic fever, malaise, joint pains, myalgias, and fatigue) means a lessening or improvement of one or more symptoms of an autoimmune disease as compared to not administering a BCL6 inhibitor. “Ameliorating” also includes shortening or reduction in duration of a symptom.

As used therein, “delaying” the development of an autoimmune disease means to defer, hinder, slow, retard, stabilize, and postpone progression of an autoimmune disease. This delay can be of varying lengths of time, depending on the history of the disease and individuals being treated. As is evident to one skilled in me art, a sufficient or significant delay can, in effect, encompass prevention, in that the individual does not develop an autoimmune disease. A method that “delays” development of a symptom is a method that reduces probability of developing the symptom in a given time frame and reduces extent of the symptoms in a given time frame, when compared to not using the method. Such comparisons are typically based on clinical studies, using a statistically significant number of subjects.

An “individual” is a vertebrate, preferably a mammal, more preferably a human. Mammals include, but are not limited to, farm animals (such as cows), sport animals, pets (such as cats, dogs and horses), primates, mice and rats.

As intended herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

### DETAILED DESCRIPTION

It has now been found that compounds that act as inhibitors of BCL6 can be useful in the treatment of autoimmune diseases. Without being bound to any particular theory, it is believed that increased Tfh activity promoted by BCL6 can lead to non-specific antibody responses and eventually to autoimmunity, while blockade of BCL6 activity can block Tfh function and thus inhibit autoimmune disease progression.

The present invention provides a new medical use for BCL6 inhibitors and pharmaceutical compositions containing them. In one aspect, the present invention provides a method for inhibiting or stopping abnormal Tfh activity in individuals suffering from an autoimmune disease, comprising administering to an individual in need of such treatment an effective amount of a BCL6 inhibitor. In this aspect, the present method is expected to halt non-specific stimulation of B cells that produce auto-antibodies and treat auto-immune disease.

In a further aspect, the present invention relates to administering an effective amount of a BCL6 inhibitor to treat one or more autoimmune disease(s).

Autoimmune disease can be divided into two categories. Organ-specific autoimmune diseases occur when the immune system targets specific cells, tissues, or organs. Generalized autoimmune diseases occur when the immune system attacks the body without discriminating among different types of tissues or target cells. Exemplary organ-specific autoimmune diseases include insulin dependent diabetes, Hashimoto's thyroiditis, Grave's disease, Pernicious anemia, Myasthenia gravis, Pemphigus vulgaris, and Crohn's disease. Exemplary generalized autoimmune diseases include Systemic lupus erythematosus (SLE), Rheumatoid arthritis, Scleroderma, Sarcoidosis, and Guillain-Barre Syndrome.

The present invention relates to treating all types of autoimmune disease, including organ-specific and general autoimmune diseases including, but not limited to, lupus erythematosus, ankylosing spondylitis, Chagas disease, chronic obstructive pulmonary disease, Crohn's Disease, dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, and Wegener's granulomatosis.

In accordance with the present invention, any BCL6 inhibitor can be used. One class of preferred BCL6 inhibitors is BCL6 peptide inhibitors (“BPIs”). Particularly preferred is the BPI of the following formula (“BPI-1”): NH₂-G(RRRQRRKKR)GG(RGIEHAAR)GG(DIM)G(EW)G(NEIF)G(AIA)G(FL)G-OH, where the amino acids are in single letter code, and the amino acids in parentheses are the D-isomer forms of the amino acids (61).

Also included in this aspect of the invention are peptides which are functionally equivalent to BPI-1 and structurally related thereto. For example, in this aspect a BPI-1 related peptide may have one or more conservative amino acid substitutions in the sequence of BPI-1 which does not substantially alter the BCL6 inhibiting activity of BPI-1. Also included in this aspect are peptides that are >70% identical, >80% identical, >90% identical; preferably >95% identical to BPI-1. As used herein, the term “conservative substitution” means amino acid substitutions that do not substantially alter the activity or binding affinity of the protein or peptide molecule. Typically, conservative amino acid substitutions involve substitution of one amino acid for another amino acid with similar chemical properties (e.g. charge or hydrophobicity). The following six groups each contain amino acids that are typical but not necessarily exclusive conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The terms “identical” or percent “identity,” in the context of two or more nucleic acids or polypeptide or peptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. With respect to the BCL6-inhibiting peptides of this invention, sequence identity is determined over the full length of a peptide, e.g. BPI-1.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information.

Example suitable non-peptide inhibitors for use according to the present invention are disclosed in WO 2008/066887, the entire contents of which are herein incorporated by reference. Among the compounds disclosed therein, those of formula “Compound 1” are preferred:

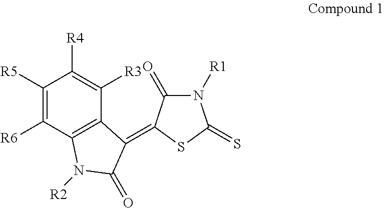

wherein R1 is selected from the group consisting of H, a hydroxy, a halogen, a formyl, an acyl, a carboxy, a keto, an amido, a carbamoyl, a guanidino, a ureido, a amidino, a nitro, an amino, a thiol, a thioether, a mercapto, a sulfinyl, a sulfonyl, a sulfonamide, a cyanide, an amidine, a carbamate, an imine, an amide, a hydrazine, an acetyl, an aminal, an alkoxy, an aryloxy, an aldehyde, an anhydride, a heteroaryl, a carboxyaryl, a fused cycloalkyl, a fused heterocyclic group, a fused aryl, a fused heteroaryl, or a C₁-C₁₀ straight or branched alkyl, carboxyalkyl, cycloalkyl, alkenyl, carboxyalkenyl, cycloalkenyl, ketone, alkynyl, carboxyalkyl, cycloalkyl, ether, cycloether, amine, nitrile, and heterocyclic group, and

R2-R6 are each independently selected from the group consisting of H, a hydroxy, a halogen, a formyl, an acyl, a carboxy, a keto, an amido, a carbamoyl, a guanidino, a ureido, a amidino, a nitro, an amino, a thiol, a thioether, a mercapto, a sulfinyl, a sulfonyl, a sulfonamide, a cyanide, an amidine, a carbamate, an imine, an amide, a hydrazine, an acetyl, an aminal, an alkoxy, an aryloxy, an aldehyde, an anhydride, an aryl, a heteroaryl, a carboxyaryl, a fused cycloalkyl, a fused heterocyclic group, a fused aryl, a fused heteroaryl, or a C₁-C₁₀ straight or branched alkyl, carboxyalkyl, cycloalkyl, alkenyl, carboxyalkenyl, cycloalkenyl, ketone, alkynyl, carboxyalkyl, cycloalkyl, ether, cycloether, amine, nitrile, or heterocyclic group, a ring encompassing any two adjacent members of R2-R6, and any combination thereof.

A particularly preferred BCL6 inhibitor is the molecule “79-6” of the following formula:

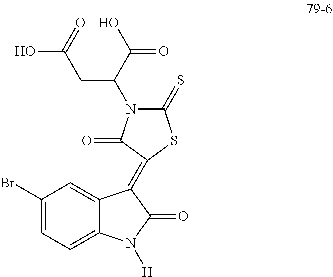

BCL6 inhibitors may be employed in accordance with the invention in free or in pharmaceutically acceptable salt form, e.g. as known in the art, for example, hydrochlorides; hydrochloride hydrates and dehydrates; and mesylates. References to BCL6 inhibitors collectively or individually throughout the present specification and claims are accordingly to be understood as embracing both free compounds and such pharmaceutically acceptable salt forms, e.g. as clinically employed, and further also solvates, e.g. hydrates, or specific crystal forms of any of these compounds or salts.

For use in accordance with the present invention, the appropriate dosage will, of course, vary depending on, for example, the particular BCL6 inhibitor employed, the mode of administration, and the nature and severity of the condition to be treated as well as the specific condition to be treated and is within the purview of the treating physician. An indicated daily dosage will typically be from about 0.05 mg/kg to about 50 mg/kg, daily to weekly, for example administered once daily, or in divided doses up to four times a day or in sustained release form.

In the case of a BPI, an appropriate dosage for administration will usually be in the range of about 0.05 mg/kg to 50 mg/kg, daily to weekly, more preferably 1 mg/kg to 35 mg/kg, daily to weekly, and most preferably 2 mg/kg to 20 mg/kg, daily to weekly. When the BCL6 inhibitor is molecule 79-6, an appropriate dosage for administration will usually be in the range of about 0.05 mg/kg to 100 mg/kg per day, more preferably 1 mg/kg to 75 mg/kg per day, and most preferably 5 mg/kg to 50 mg/kg per day.

For use in accordance with the present invention, BCL6 inhibitors may be administered by any conventional route, in particular: enterally, orally, pulmonarily, or nasally, e.g. in the form of tablets or capsules, sprays, via suppositories, or parenterally, e.g. in the form of injectable solutions or suspensions, for intravenous, intra-muscular, sub-cutaneous, or intra-peritoneal injection. Administration of a BPI as an injectable solution is particularly preferred.

Administration of a BCL6 inhibitor is expected to treat autoimmune disease(s) by inhibiting or reducing the production of one or more autoantibodies associated with a particular autoimmune disease. In some cases, a simple blood test or microarray method can be applied to detect the autoantibody(ies) before and/or after treatment ensues as a means for assessing disease progression and/or the efficacy of treatment. For example, thyroid antibodies can be detected using available blood tests to monitor Hashimoto's thyroiditis.

Suitable formulations and pharmaceutical compositions for use in accordance with the present invention will include those formulated in a conventional manner using one or more pharmaceutically acceptable carriers or excipients, and any of those known and commercially available and currently employed in the clinical setting. Thus, the compounds for use in accordance with the present invention may be formulated for oral, buccal, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound over an extended period. For buccal administration the compositions may take the form of tablets or lozenges formulated in a conventional manner known to the skilled artisan.

Compounds for use according to the present invention may also be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the active ingredient may be in powder form for constitution with a suitable vehicle, e.g. sterile pyrogen-free water, before use.

The compounds for use according to the present invention may also be formulated for rectal administration as suppositories or retention enemas, e.g. containing conventional suppository bases such as cocoa butter or other glycerides.

The invention has been described with reference to various illustrative embodiments and techniques. However, it should be understood that many variations and modifications, as are known in the art, may be made while remaining within the scope of the claimed invention. The examples that follow are illustrative and are not intended to be limiting.

### EXAMPLE 1

### BCL6 can Promote Tfh Gene Expression in Vitro

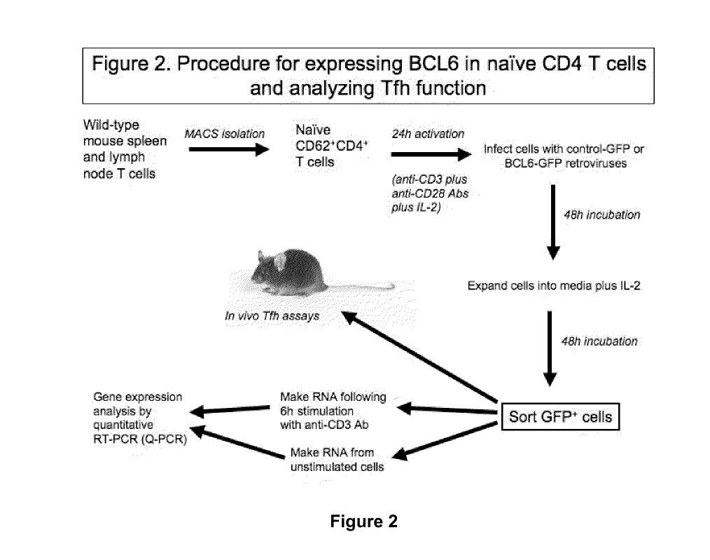

Since BCL6 was found to be critically involved in Tfh cell development, the forced expression of BCL6 in CD4 T cells was tested to establish whether it could promote Tfh gene expression in vitro. BCL6 was markedly down-regulated following the activation of CD4 T cells, and thus infecting T cells with a BCL6-expressing RV led to greatly increased BCL6 expression over background (33, 50). The RV-infection procedure that was used for expressing BCL6 in T cells is shown in FIG. 2. This procedure resulted in BCL6 being expressed in the T cells for 5 days prior to cell sorting and analysis, and is similar to what was used in Johnson et al (4). We hypothesize that this 5 day period allows time for BCL6 to program Tfh gene expression. After sorting of GFP-expressing T cells, gene expression was assessed following activation of the cells for 6 hours with plate-bound anti-CD3 Ab.

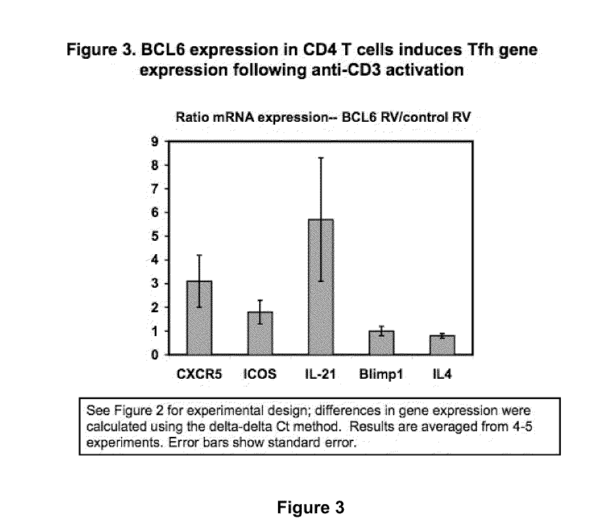

As shown in FIG. 3, BCL6-expressing cells clearly had a Tfh-like phenotype as assessed by increased expression of CXCR5, ICOS and IL-21. In particular, the up-regulation of IL-21 by BCL6 was very strong, almost 20-fold in the experiment shown (FIG. 3). In three experiments, the average induction of IL-21 by BCL6 expression was 27-fold (data not shown). Blimp1 and IL-4, target genes of BCL6 in T cells, were not markedly repressed under CD3-stimulated conditions (FIG. 3). On the other hand, Blimp1 and IL-4 expression was not increased by BCL6, thus showing specificity to the stimulatory action of BCL6 on CXCR5, ICOS and IL-21 expression. In the absence of CD3 stimulation, BCL6 actually weakly repressed all CXCR5, ICOS and IL-21 but strongly repressed Blimp1 and IL-4 (data not shown). Without being bound to any particular theory, these data indicate that BCL6 appears to regulate steady-state transcription in a different manner than transcription immediately following T cell activation.

### EXAMPLE 2

### Treatment of Lupus Erythematosus

The individual is a 30 year old female with symptoms including chronic fever, malaise, joint pains, myalgias, fatigue, indicative of lupus. The individual has previously been treated, largely unsuccessfully, with cyclophosphamides, corticosteroids and immunosuppressants.

The individual is treated for 1 to 60 days sequentially with BPI-1 administered at a dose of 4 mg/kg daily. Treatment results in long-lasting amelioration of lupus with a reduction in the severity and frequency of flares and their symptoms.

Equivalent results are obtainable in equivalent or comparable trials with individuals exhibiting similar symptomatology by employing BCL6 inhibitors other than BPI, for example by using molecule 79-6. Similar results are also achievable employing BCL6 inhibitors in clinical trials involving subjects exhibiting other autoimmune diseases.

### REFERENCES

1. Fazilleau, N., L. Mark, L. J. McHeyzer-Williams, and M. G. McHeyzer-Williams. 2009. Follicular helper T cells: lineage and location. Immunity 30:324-335.

2. Fazilleau, N., L. J. McHeyzer-Williams, H. Rosen, and M. G. McHeyzer-Williams. 2009. The function of follicular helper T cells is regulated by the strength of T cell antigen receptor binding. Nat Immunol 10:375-384.

3. Chtanova, T., S. G. Tangye, R. Newton, N. Frank, M. R. Hodge, M. S. Rolph, and C. R. Mackay. 2004. T follicular helper cells express a distinctive transcriptional profile, reflecting their role as non-Th1/Th2 effector cells that provide help for B cells. J Immunol 173:68-78.

4. Johnston, R. J., A. C. Poholek, D. DiToro, I. Yusuf, D. Eto, B. Barnett, A. L. Dent, J. Craft, and S. Crotty. 2009. Bcl6 and Blimp-1 are reciprocal and antagonistic regulators of T follicular helper cell differentiation. Science 325:1006-1010.

5. Nurieva, R. I., Y. Chung, G. J. Martinez, X. 0. Yang, S. Tanaka, T. D. Matskevitch, Y. H. Wang, and C. Dong. 2009. Bcl6 mediates the development of T follicular helper cells. Science 325:1001-1005.

6. Yu, D., S. Rao, L. M. Tsai, S. K. Lee, Y. He, E. L. Sutcliffe, M. Srivastava, M. Linterman, L. Zheng, N. Simpson, J. I. Ellyard, I. A. Parish, C. S. Ma, Q. J. Li, C. R. Parish, C. R. Mackay, and C. G. Vinuesa. 2009. The Transcriptional Repressor Bcl-6 Directs T Follicular Helper Cell Lineage Commitment. Immunity.

7. Nurieva, R. I., Y. Chung, D. Hwang, X. O. Yang, H. S. Kang, L. Ma, Y. H. Wang, S. S. Watowich, A. M. Jetten, Q. Tian, and C. Dong. 2008. Generation of T follicular helper cells is mediated by interleukin-21 but independent of T helper 1, 2, or 17 cell lineages. Immunity 29:138-149.

8. Breitfeld, D., L. Ohl, E. Kremmer, J. Ellwart, F. Sallusto, M. Lipp, and R. Forster. 2000. Follicular B helper T cells express CXC chemokine receptor 5, localize to B cell follicles, and support immunoglobulin production. J Exp Med 192:1545-1552.

9. Schaerli, P., K. Willimann, A. B. Lang, M. Lipp, P. Loetscher, and B. Moser. 2000. CXC chemokine receptor 5 expression defines follicular homing T cells with B cell helper function. J Exp Med 192:1553-1562.

10. Vinuesa, C. G., M. C. Cook, C. Angelucci, V. Athanasopoulos, L. Rui, K. M. Hill, D. Yu, H. Domaschenz, B. Whittle, T. Lambe, I. S. Roberts, R. R. Copley, J. I. Bell, R. J. Gornall, and C. C. Goodnow. 2005. A RING-type ubiquitin ligase family member required to repress follicular helper T cells and autoimmunity. Nature 435:452-458.

11. McHeyzer-Williams, L. J., N. Pelletier, L. Mark, N. Fazilleau, and M. G. McHeyzer-Williams. 2009. Follicular helper T cells as cognate regulators of B cell immunity. Curr Opin Immunol.

12. Ettinger, R., G. P. Sims, A. M. Fairhurst, R. Robbins, Y. S. da Silva, R. Spolski, W. J. Leonard, and P. E. Lipsky. 2005. IL-21 induces differentiation of human naive and memory B cells into antibody-secreting plasma cells. J Immunol 175:7867-7879.

13. Kuchen, S., R. Robbins, G. P. Sims, C. Sheng, T. M. Phillips, P. E. Lipsky, and R. Ettinger. 2007. Essential role of IL-21 in B cell activation, expansion, and plasma cell generation during CD4+T cell-B cell collaboration. J Immunol 179:5886-5896.

14. Dienz, 0., S. M. Eaton, J. P. Bond, W. Neveu, D. Moquin, R. Noubade, E. M. Briso, C. Charland, W. J. Leonard, G. Ciliberto, C. Teuscher, L. Haynes, and M. Rincon. 2009. The induction of antibody production by IL-6 is indirectly mediated by IL-21 produced by CD4+T cells. J Exp Med 206:69-78.

15. Ozaki, K., R. Spolski, R. Ettinger, H. P. Kim, G. Wang, C. F. Qi, P. Hwu, D. J. Shaffer, S. Akilesh, D. C. Roopenian, H. C. Morse, 3rd, P. E. Lipsky, and W. J. Leonard. 2004. Regulation of B cell differentiation and plasma cell generation by IL-21, a novel inducer of Blimp-1 and Bcl-6. J Immunol 173:5361-5371.

16. Vogelzang, A., H. M. McGuire, D. Yu, J. Sprent, C. R. Mackay, and C. King. 2008. A fundamental role for interleukin-21 in the generation of T follicular helper cells. Immunity 29:127-137.

17. Linterman, M. A., R. J. Rigby, R. K. Wong, D. Yu, R. Brink, J. L. Cannons, P. L. Schwartzberg, M. C. Cook, G. D. Walters, and C. G. Vinuesa. 2009. Follicular helper T cells are required for systemic autoimmunity. J Exp Med 206:561-576.

18. Hu, Y. L., D. P. Metz, J. Chung, G. Siu, and M. Zhang. 2009. B7RP-1 blockade ameliorates autoimmunity through regulation of follicular helper T cells. J Immunol 182:1421-1428.

19. Odegard, J. M., B. R. Marks, L. D. DiPlacido, A. C. Poholek, D. H. Kono, C. Dong, R. A. Flavell, and J. Craft. 2008. ICOS-dependent extrafollicular helper T cells elicit IgG production via IL-21 in systemic autoimmunity. J Exp Med 205:2873-2886.

20. Leonard, W. J., R. Zeng, and R. Spolski. 2008. Interleukin 21: a cytokine/cytokine receptor system that has come of age. J Leukoc Biol 84:348-356.

21. Herber, D., T. P. Brown, S. Liang, D. A. Young, M. Collins, and K. Dunussi-Joannopoulos. 2007. IL-21 has a pathogenic role in a lupus-prone mouse model and its blockade with IL-21 R.Fc reduces disease progression. J Immunol 178:3822-3830.

22. Seyfert, V. L., D. Allman, Y. He, and L. M. Staudt. 1996. Transcriptional repression by the proto-oncogene BCL-6. Oncogene 12:2331-2342.

23. Ye, B. H., F. Lista, F. Lo Coco, D. M. Knowles, K. Offit, R. S. Chaganti, and R. Dalla-Favera. 1993. Alterations of a zinc finger-encoding gene, BCL-6, in diffuse large-cell lymphoma. Science 262:747-750.

24. Chang, C. C., B. H. Ye, R. S. Chaganti, and R. Dalla-Favera. 1996. BCL-6, a POZ/zincfinger protein, is a sequence-specific transcriptional repressor. Proc Natl Acad Sci USA 93:6947-6952.

25. Dhordain, P., 0. Albagli, R. J. Lin, S. Ansieau, S. Quief, A. Leutz, J. P. Kerckaert, R. M. Evans, and D. Leprince. 1997. Corepressor SMRT binds the BTB/POZ repressing domain of the LAZ3/BCL6 oncoprotein. Proc Natl Acad Sci U S A 94:10762-10767.

26. Huynh, K. D., W. Fischle, E. Verdin, and V. J. Bardwell. 2000. BCoR, a novel corepressor involved in BCL-6 repression. Genes Dev 14:1810-1823.

27. Huynh, K. D., and V. J. Bardwell. 1998. The BCL-6 POZ domain and other POZ domains interact with the co- repressors N-CoR and SMRT. Oncogene 17:2473-2484.

28. Zhang, H., S. Okada, M. Hatano, S. Okabe, and T. Tokuhisa. 2001. A new functional domain of Bcl6 family that recruits histone deacetylases. Biochim Biophys Acta 1540:188-200.

29. Onizuka, T., M. Moriyama, T. Yamochi, T. Kuroda, A. Kazama, N. Kanazawa, K. Sato, T. Kato, H. Ota, and S. Mori. 1995. BCL-6 gene product, a 92- to 98-kD nuclear phosphoprotein, is highly expressed in germinal center B cells and their neoplastic counterparts. Blood 86:28-37.

30. Cattoretti, G., C. C. Chang, K. Cechova, J. Zhang, B. H. Ye, B. Falini, D. C. Louie, K. Offit, R. S. Chaganti, and R. Dalla-Favera. 1995. BCL-6 protein is expressed in germinal center B cells. Blood 86:45-53.

31. Allman, D., A. Jain, A. Dent, R. R. Maile, T. Selvaggi, M. R. Kehry, and L. M. Staudt. 1996. BCL-6 expression during B-cell activation. Blood 87:5257-5268.

32. Lund, R. J., E. K. Ylikoski, T. Aittokallio, O. Nevalainen, and R. Lahesmaa. 2003. Kinetics and STAT4- or STATE-mediated regulation of genes involved in lymphocyte polarization to Th1 and Th2 cells. Eur J Immunol 33:1105-1116.

33. Dent, A. L. unpublished data.

34. Staudt, L. M., A. L. Dent, A. L. Shaffer, and X. Yu. 1999. Regulation of lymphocyte cell fate decisions and lymphomagenesis by BCL-6. Intern Rev Immunol 18:381.

35. Dalla-Favera, R., A. Migliazza, C. C. Chang, H. Niu, L. Pasqualucci, M. Butler, Q. Shen, and G. Cattoretti. 1999. Molecular pathogenesis of B cell malignancy: the role of BCL-6. Curr Top Microbiol Immunol 246:257-263.

36. Pasqualucci, L., 0. Bereschenko, H. Niu, U. Klein, K. Basso, R. Guglielmino, G. Cattoretti, and R. Dalla-Favera. 2003. Molecular pathogenesis of non-Hodgkin's lymphoma: the role of Bcl-6. Leuk Lymphoma 44 Suppl 3:S5-12.

37. Kusam, S., and A. Dent. 2007. Common mechanisms for the regulation of B cell differentiation and transformation by the transcriptional repressor protein BCL-6. Immunol Res 37:177-186.

38. Kusam, S., V. Munugalavadla, D. Sawant, and A. Dent. 2009. BCL6 cooperates with CD40 stimulation and loss of p53 function to rapidly transform primary B cells. Int J Cancer 125:977-981.

39. Dent, A. L., A. L. Shaffer, X. Yu, D. Allman, and L. M. Staudt. 1997. Control of inflammation, cytokine expression, and germinal center formation by BCL-6. Science 276:589-592.

40. Gupta, S., M. Jiang, A. Anthony, and A. B. Pernis. 1999. Lineage-specific modulation of interleukin 4 signaling by interferon regulatory factor 4. J Exp Med 190:1837-1848.

41. Harris, M. B., C. C. Chang, M. T. Berton, N. N. Danial, J. Zhang, D. Kuehner, B. H. Ye, M. Kvatyuk, P. P. Pandolfi, G. Cattoretti, R. Dalla-Favera, and P. B. Rothman. 1999. Transcriptional Repression of Stat6-Dependent Interleukin-4-Induced Genes by BCL-6: Specific Regulation of !epsilon Transcription and Immunoglobulin E Switching. Mol Cell Biol 19:7264-7275.

42. Mendez, L. M., J. M. Polo, J. J. Yu, M. Krupski, B. B. Ding, A. Melnick, and B. H. Ye. 2008. CtBP is an essential corepressor for BCL6 autoregulation. Mol Cell Biol 28:2175-2186.

43. Phan, R. T., and R. Dalla-Favera. 2004. The BCL6 proto-oncogene suppresses p53 expression in germinal-centre B cells. Nature 432:635-639.

44. Phan, R. T., M. Saito, K. Basso, H. Niu, and R. Dalla-Favera. 2005. BCL6 interacts with the transcription factor Miz-1 to suppress the cyclin-dependent kinase inhibitor p21 and cell cycle arrest in germinal center B cells. Nat Immunol 6:1054-1060.

45. Shaffer, A. L., X. Yu, Y. He, J. Boldrick, E. P. Chan, and L. M. Staudt. 2000. BCL-6 represses genes that function in lymphocyte differentiation, inflammation, and cell cycle control. Immunity 13:199-212.

46. Ci, W., J. M. Polo, L. Cerchietti, R. Shaknovich, L. Wang, S. N. Yang, K. Ye, P. Farinha, D. E. Horsman, R. D. Gascoyne, 0. Elemento, and A. Melnick. 2009. The BCL6 transcriptional program features repression of multiple oncogenes in primary B cells and is deregulated in DLBCL. Blood 113:5536-5548.

47. Yu, R. Y., X. Wang, F. J. Pixley, J. J. Yu, A. L. Dent, H. E. Broxmeyer, E. R. Stanley, and B. H. Ye. 2005. BCL-6 negatively regulates macrophage proliferation by suppressing autocrine IL-6 production. Blood 105:1777-1784.

48. Toney, L. M., G. Cattorretti, J. A. Graf, T. Merghoub, P. P. Pandolfi, R. Dalla-Favera, B. H. Ye, and A. L. Dent. 2000. BCL-6 regulates chemokine gene transcription in macrophages. Nat. Immunol. 1:214-220.

49. Takeda, N., M. Arima, N. Tsuruoka, S. Okada, M. Hatano, A. Sakamoto, Y. Kohno, and T. Tokuhisa. 2003. Bcl6 is a transcriptional repressor for the IL-18 gene. J Immunol 171:426-431.

50. Kusam, S., L. M. Toney, H. Sato, and A. L. Dent. 2003. Inhibition of Th2 differentiation and GATA-3 expression by BCL-6. J Immunol 170:2435-2441.

51. Arima, M., H. Toyama, H. Ichii, S. Kojima, S. Okada, M. Hatano, G. Cheng, M. Kubo, T. Fukuda, and T. Tokuhisa. 2002. A putative silencer element in the IL-5 gene recognized by Bcl6. J Immunol 169:829-836.

52. Cimmino, L., G. A. Martins, J. Liao, E. Magnusdottir, G. Grunig, R. K. Perez, and K. L. Calame. 2008. Blimp-1 attenuates Th1 differentiation by repression of ifng, tbx21, and bcl6 gene expression. J Immunol 181:2338-2347.

53. Yoshida, K., A. Sakamoto, K. Yamashita, E. Arguni, S. Horigome, M. Arima, M. Hatano, N. Seki, T. Ichikawa, and T. Tokuhisa. 2006. Bcl6 controls granzyme B expression in effector CD8+T cells. Eur J Immunol 36:3146-3156.

54. Ye, B. H., G. Cattoretti, Q. Shen, J. Zhang, N. Hawe, R. de Waard, C. Leung, M. Nouri-Shirazi, A. Orazi, R. S. Chaganti, P. Rothman, A. M. Stall, P. P. Pandolfi, and R. Dalla-Favera. 1997. The BCL-6 proto-oncogene controls germinal-centre formation and Th2-type inflammation. Nat Genet 16:161-170.

55. Dent, A. L., J. Hu-Li, W. E. Paul, and L. S. Staudt. 1998. T helper type 2 inflammatory disease in the absence of IL-4 and STATE. Proc Natl Acad Sci U S A 95:13823-13828.

56. Adra, C. N., P. S. Gao, X. Q. Mao, B. W. Baron, S. Pauker, T. Miki, T. Shirakawa, and J. M. Hopkin. 1998. Variants of B cell lymphoma 6 (BCL6) and marked atopy. Clin Genet 54:362-364.

57. Peng, S. L., M. P. Madaio, D. P. Hughes, I. N. Crispe, M. J. Owen, L. Wen, A. C. Hayday, and J. Craft. 1996. Murine lupus in the absence of alpha beta T cells. J Immunol 156:4041-4049.

58. Merino, R., M. Iwamoto, L. Fossati, and S. Izui. 1993. Polyclonal B cell activation arises from different mechanisms in lupus-prone (NZB x NZW)F1 and MRL/MpJ-Ipr/Ipr mice. J Immunol 151:6509-6516.

59. Yu, D., A. H. Tan, X. Hu, V. Athanasopoulos, N. Simpson, D. G. Silva, A. Hutloff, K. M. Giles, P. J. Leedman, K. P. Lam, C. C. Goodnow, and C. G. Vinuesa. 2007. Roquin represses autoimmunity by limiting inducible T-cell co-stimulator messenger RNA. Nature 450:299-303.

60. Dent, A. L., J. Yewdell, F. Puvion-Dutilleul, M. H. Koken, H. de The, and L. M. Staudt. 1996. LYSP100-associated nuclear domains (LANDs): description of a new class of subnuclear structures and their relationship to PML nuclear bodies. Blood 88:1423-1426.

61. Cerchietti, L. C., S. N. Yang, R. Shaknovich, K. Hatzi, J. M. Polo, A. Chadburn, S. F. Dowdy, and A. Melnick. 2009. A peptomimetic inhibitor of BCL6 with potent antilymphoma effects in vitro and in vivo. Blood 113:3397-3405.

62. Appleby, P., G. Telford, and G. B. Naylor. 1993. Suppression of autoimmune disease in NZB/W F1 mice by treatment with the novel immunomodulator BTS 63155. Clin Exp Immunol 93:313-317.

63. Cohen, M. G., K. M. Pollard, and L. Schrieber. 1988. Relationship of age and sex to autoantibody expression in MRL-+/+ and MRL-Ipr/Ipr mice: demonstration of an association between the expression of antibodies to histones, denatured DNA and Sm in MRL-+/+ mice. Clin Exp Immunol 72:50-54.

64. Drake, C. G., S. J. Rozzo, T. J. Vyse, E. Palmer, and B. L. Kotzin. 1995. Genetic contributions to lupus-like disease in (NZB x NZW)F1 mice. Immunol Rev 144:51-74.

65. O′Dell, J. R., and B. L. Kotzin. 1985. In vitro production of anti-histone antibodies by spleen cells from young autoantibody negative NZB/NZW mice. J Immunol 135:1101-1107.

66. Nose, M., M. Nishihara, J. Kamogawa, M. Terada, and S. Nakatsuru. 2000. Genetic basis of autoimmune disease in MRL/Ipr mice: dissection of the complex pathological manifestations and their susceptibility loci. Rev Immunogenet 2:154-164.

67. Elouaai, F., J. Lule, H. Benoist, S. Appolinaire-Pilipenko, C. Atanassov, S. Muller, and G. J. Fournie. 1994. Autoimmunity to histones, ubiquitin, and ubiquitinated histone H2A in NZB x NZW and MRL-Ipr/Ipr mice.

Anti-histone antibodies are concentrated in glomerular eluates of lupus mice. Nephrol Dial Transplant 9:362-366.

68. William, J., C. Euler, S. Christensen, and M. J. Shlomchik. 2002. Evolution of autoantibody responses via somatic hypermutation outside of germinal centers. Science 297:2066-2070.

69. Poholek A.C., Hansen K., Hernandez S.G., Eto D., Chandele A., Weinstein J.S., Dong X., Odegard J.M., Kaech S.M., Dent A.L., Crotty S, Craft J.J. (2010). In vivo regulation of Bcl6 and T follicular helper cell development. Immunol. 2010 Jul 1;185(1):313-26. Epub 2010 Jun 2.

1. A method for inhibiting or stopping abnormal Tfh activity in individuals suffering from an autoimmune disease, comprising administering to an individual in need of such treatment an effective amount of a BCL6 inhibitor.

2. The method of claim 1, wherein the autoimmune disease is selected from the group consisting of lupus erythematosus, ankylosing spondylitis, Chagas disease, chronic obstructive pulmonary disease, Crohns Disease, dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and combinations thereof.

3. The method of claim 1, wherein the autoimmune disease is lupus erythematosus.

4. The method of claim 1, wherein said BCL6 inhibitor is a peptide or non-peptide inhibitor.

5. The method of claim 4, wherein said peptide inhibitor is a BPI.

6. The method of claim 5, wherein the BPI is BPI-1.

7. The method of claim 1, wherein the BCL6 inhibitor is a molecule of formula Compound 1.

8. The method of claim 1, wherein the BCL6 inhibitor is molecule 79-6.

9. The method of claim 1 wherein said BCL6 inhibitor is administered once daily to said individual in an amount of about 0.05 mg/kg to about 50 mg/kg.

10. A method of treatment of an autoimmune disease in an individual in need thereof, comprising administering an effective amount of a BCL6 inhibitor to inhibit non-specific stimulation of B cells thereby ameliorating autoimmune disease symptoms in the individual.

11. The method of claim 10 wherein said BCL6 inhibitor is a peptide or non-peptide inhibitor.

12. The method of claim 11 wherein said peptide inhibitor is BPI-1.

13. The method of claim 10, wherein the BCL6 inhibitor is a molecule of formula Compound 1.

14. The method of claim 10 wherein said BCL6 inhibitor is molecule 79-6.

15. The method of claim 10 wherein said BCL6 inhibitor is administered once daily to said individual in an amount of about 0.05 mg/kg to about 50 mg/kg.

16. The method of claim 10 wherein said treatment results in reduced secretion of auto-antibodies by B cells in said individual.

17. The method of claim 10 further comprising the step of assessing treatment efficacy by detecting the level of one or more autoantibodies in blood from said individual before and after said treatment ensues.

18. The method of claim 10 wherein the autoimmune disease is selected from lupus erythematosus, ankylosing spondylitis, Chagas disease, chronic obstructive pulmonary disease, Crohns Disease, dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis, sarcoidosis, schizophrenia, scleroderma, Sjögren's syndrome, stiff person syndrome, temporal arteritis, ulcerative colitis, vasculitis, vitiligo, Wegener's granulomatosis, and combinations thereof.

19. The method of claim 10 wherein the autoimmune disease is lupus erythematosus.

20. A method of treatment of an autoimmune disease, comprising administering an effective amount of a BCL6 inhibitor to an individual in need thereof.
